(19) United States
(12) Patent Application Publication
(10) Pub. No.: US 2026/0325789 A1
(43) Pub. Date: Oct. 22, 2026

(54) HANDLE ASSEMBLY FOR CATHETER BLOOD PUMPS

(71) Applicant: Shifamed Holdings, LLC, Campbell, CA (US)

(72) Inventor: Daniel VARGHAI, Scotts Valley, CA (US)

(21) Appl. No.: 19/113,327

(22) PCT Filed: Sep. 20, 2023
(86) PCT No.: PCT/US2023/074709

§ 371 Date: Mar. 19, 2025

Related U.S. Application Data
(60) Provisional application No. 63/376,374, filed on Sep. 20, 2022.

(51) Int. Cl. A61M 25/00; A61M 60/13; A61M 60/237; A61M 60/414; A61M 60/808

(52) U.S. Cl. CPC A61M 25/0014; A61M 60/13; A61M 60/237; A61M 60/414; A61M 60/808; A61M 25/0097

(57) ABSTRACT

Catheter blood pumps are provided that include a catheter shaft extending through a lumen of a catheter. The catheter shaft can have a proximal end and a distal end, where the distal end can be operably coupled to an intravascular impeller locatable within a vessel. A handle can comprise a sealed housing, a motor in operable communication with the catheter shaft proximal end, and a strain relief disposed at least partially in the handle and providing access for the catheter shaft to pass from an interior of the handle to an exterior of the handle. A carrier assembly is also provided for coupling the motor to the handle. Methods of use are also provided.

### CROSS REFERENCE TO RELATED APPLICATIONS

This patent application claims priority to U.S. provisional patent application No. 63/376,374, titled “CATHETER BLOOD PUMPS,” and filed on Sep. 20, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. Intra-aortic balloon pumps (IABP) are used to support circulatory function, such as treating heart failure patients. An IABP is typically placed within the aorta, and inflated and deflated in counter-pulsation fashion with the heart contractions, with one function being to provide additive support to the circulatory system. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

More recently, minimally invasive rotary blood pumps have been developed, which are inserted into the body in connection with the cardiovascular system to pump arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient. The smallest rotary blood pumps currently available may be percutaneously inserted into the vasculature of a patient through an access sheath, thereby avoiding more extensive surgical intervention, or through a vascular access graft. One such device is a percutaneously inserted ventricular support device.

Although blood pumps exist, there is a need to provide additional improvements in the field of ventricular support devices and similar blood pumps for treating compromised cardiac blood flow.

### SUMMARY OF THE DISCLOSURE

The disclosure is related to intravascular blood pump and methods of their use.

In general, an intravascular blood pump system can comprise a catheter shaft extending through a lumen of a catheter. The catheter shaft can have a proximal end and a distal end, where the distal end can be operably coupled to an intravascular impeller locatable within a vessel. A handle can comprise a sealed housing, a motor in operable communication with the catheter shaft proximal end, and a fluid pump operably coupled to the motor.

In some examples, the catheter shaft proximal end can be removably coupled to a rigid member extending through a sealed strain relief at a distal end of the handle. In some examples, the system can further comprise a cradle assembly locatable around an exterior surface of the motor within the handle, the cradle assembly can have one or more surfaces configured to retain a circuit board thereon.

In some examples, a cradle assembly comprises one or more routing channels that can be configured to route one or more tubes within the sealed housing. In some examples, the catheter shaft proximal end can be configured to engage a ball seal connector at a distal end of the rigid member. In some examples, the system can further comprise a one or more conduits extending through a proximal strain relief configured to transition one or more electrical connections to a circuit board on the cradle assembly.

In some examples, the system can further comprise a catheter introducer having a proximal end configured to axially engage a sheath hub. In some examples, the catheter can be a multi-layer catheter comprising at least one braid layer, the brain layer comprising one or more braid angles configured to conserve rotation from a proximal end of the multi-layer catheter to a distal end of the multi-layer catheter. In some examples, the housing can be fluid-sealed.

In some examples, the system can further comprise one or more sensors operably connected to a circuit board locatable on the cradle assembly. In some examples, the system can further comprise a pressure sensor configured to determine pressure of a fluid passing through the intravascular pump system. In some examples, the system can further comprise a flow sensor configured to determine a flow rate of a fluid within the intravascular pump system.

In some examples, the system can further comprise a data transmission module configured to transfer data obtained within the system to one or more peripheral devices.

In general, circuit board carrier assembly can comprise a cradle having one or more mounting surfaces configured to receive one or more circuit boards, and one or more routing channels configured to route conduit therethrough, wherein the cradle is couplable to a motor assembly.

In some examples, the cradle can be locatable within a handle of an intravascular blood pump system. In some examples, the routing channels comprising an attachment mechanism configured to retain the conduit. In some examples, the cradle can be releasably coupled to an exterior surface of the motor assembly. In some examples, the circuit board carrier assembly can further comprise a curved inner surface, wherein the motor assembly can be generally cylindrical.

In some examples, the cradle frictionally positioned on an exterior surface of the motor assembly. In some examples, the circuit board carrier assembly can further comprise a sealed housing having a proximal strain relief and a distal tapered strain relief configured to transition conduit from an exterior of the sealed housing to the cradle within the sealed housing.

In some examples, the circuit board carrier assembly can further comprise one or more attachment elements configured to engage the interior of the sealed housing.

In some examples, the circuit board carrier assembly can further comprise the motor assembly, wherein the cradle is coupled to the motor assembly and both the motor assembly and cradle are removable from within the sealed housing.

In general, a method of operating a self-expanding blood pump within a patient can comprise the steps of inserting a compressed self-expanding blood pump within a sheathing sleeve through a proximal opening of an introducer sheath, the introducer sheath extending from its proximal opening disposed outside the patient distally into a blood vessel of the patient. Then, advancing the sheathing sleeve within the introducer sheath until a distal opening of the sheathing sleeve can be at or beyond a distal opening of the introducer sheath. Then advancing the self-expanding blood pump out of the sheathing sleeve to an expanded configuration at a deployment site within the blood vessel. Then rotating one or more impellers of the self-expanding blood pump with a motor enclosed in a handle body, wherein a drive cable can be operably coupled at a distal end to the one or more impellers and operably coupled at a proximal to the motor.

In some examples, the method can further comprise positioning the self-expanding blood pump within the blood vessel by rotating the handle body, wherein a drive cable catheter can be configured to rotate the self-expanding blood pump correspondingly to rotation of the handle body.

In some examples, the drive cable extends through a multi-layer drive cable catheter coupled to a distal end of the handle body, the multi-layer drive cable catheter configured to maintain even torque from the handle body to the self-expanding blood pump.

In some examples, the method can further comprise detecting a flow rate of a fluid from the blood pump to a motor assembly within the handle housing, wherein a one or more sensors can be configured to detect the flow rate.

In some aspects, a catheter handle device is provided, comprising a handle body; a strain relief assembly partially disposed within the handle body, the strain relief assembly providing access for an elongate member to pass from an interior of the handle body to an exterior of the handle body, the strain relief assembly comprising a coupling member that is attached to the handle body and a strain relief member attached to the coupling member and extending from the handle body, wherein the strain relief member is more flexible than the coupling member.

In some aspects, the strain relief member comprises a first polymer and the coupling member comprises a second polymer.

In some aspects, the strain relief member is molded over a distal portion of the coupling member.

In other aspects, the coupling member comprises a first coupling portion and a second coupling portion separated by a gap. In one aspect, the gap of the coupling member is configured to receive a tab or extension of the handle body.

In some aspects, the strain relief assembly is rotatable within the handle body when the tab or extension of the handle body is inserted into the gap of the coupling member. In some aspects, the gap includes an inner surface that is disc-shaped to facilitate rotation of the strain relief assembly.

In one aspect, the strain relief member forms a fluidic seal with an interior of the handle body. In some aspects, the strain relief member includes one or more sealing elements. In other aspects, the sealing elements comprise raised portions or o-rings.

In some aspects, a lumen of the coupling member is in communication with a lumen of the strain relief member.

In other aspects, the strain relief member is a flexible or compliant material and the coupling member is a substantially rigid material.

In one aspect, the elongate member comprises a catheter shaft.

In some aspects, the elongate member comprises a drive cable of a motor assembly.

In another aspect, the handle body comprises a clamshell design with a first handle body portion coupled to a second handle body portion.

In some aspects, an o-ring is provided between the first and second handle body portions to fluidically seal the handle body portion.

In another aspect, the handle body further comprises one or more mounting elements configured to support a motor assembly.

In some aspects, the one or more mounting elements comprises a first mounting element configured to support a distal portion of the motor assembly and a second mounting element configured to support a proximal portion of the motor assembly.

In another aspect, the device includes a motor assembly disposed in the handle body and supported by the one or more mounting elements.

In some aspects, one or more o-rings disposed around the motor assembly and configured to contact the one or more mounting elements to reduce vibration imparted by the motor assembly on the handle body.

In some aspects, a cradle assembly is removably coupled to the motor assembly, the cradle assembly comprising at least one printed circuit board (PCB) disposed on the cradle assembly.

In another aspect, the cradle assembly is configured to at least partially conform to an outer surface of the motor assembly.

In some aspects, the PCB further comprises electronics configured to control operation of the motor assembly.

In another aspect, an electrical connection is included on the motor assembly configured to be electrically coupled to the PCB.

In some aspects, the electrical connection passes through an opening on the cradle assembly.

In some aspects, the strain relief assembly and the coupling assembly are co-molded polymers.

A catheter blood pump handle assembly is provided, comprising a handle body; a motor assembly disposed within the handle body; a strain relief assembly partially disposed within the handle body, the strain relief assembly providing access for a drive cable to pass from the motor assembly to a catheter blood pump external to the handle body, the strain relief assembly comprising a coupling member that is attached to the handle body and a strain relief member attached to the coupling member that extends from the handle body and forms a fluidic seal with the handle body.

In one aspect, the catheter blood pump handle assembly includes a cradle assembly removably coupled to the motor assembly, the cradle assembly comprising at least one printed circuit board (PCB) disposed on the cradle assembly.

In some aspects, the cradle assembly is configured to at least partially conform to an outer surface of the motor assembly.

In other aspects, the PCB further comprises electronics configured to control operation of the motor assembly.

In one aspect, an electrical connection on the motor assembly is configured to be electrically coupled to the PCB.

In some aspects, the electrical connection passes through an opening on the cradle assembly.

In some aspects, the strain relief member comprises a first polymer and the coupling member comprises a second polymer.

In one aspect, the strain relief assembly is molded over a distal portion of the coupling member.

In one aspect, the strain relief member and the coupling member comprise co-molded polymers.

In other aspects, the strain relief member is more compliant than the coupling member.

In some aspects, the coupling member comprises a first coupling portion and a second coupling portion separated by a gap. In some aspects, the gap of the coupling member is configured to receive a tab or extension of the handle body.

In one aspect, the strain relief assembly is rotatable within the handle body when the tab or extension of the handle body is inserted into the gap.

In one aspect, the gap includes an inner surface that is disc-shaped to facilitate rotation of the strain relief assembly.

In another aspect, the strain relief member includes one or more sealing elements.

In some aspects, the sealing elements comprise raised portions or o-rings.

In another aspect, a lumen of the coupling member is in communication with a lumen of the strain relief member.

In some aspects, the strain relief member is a flexible or compliant material and the coupling member is a substantially rigid material.

A circuit board carrier assembly is provided, comprising a frame dimensioned and configured to receive a rotational motor assembly, one or more exterior mounting surfaces on the frame configured to receive one or more printed circuit boards (PCBs), and one or more openings in the frame configured to route a wire therethrough to an electrical connection of the rotational motor assembly.

In some aspects, the circuit board carrier assembly includes one or more attachment mechanisms on the frame configured to be removably coupled to a mounting bracket of the rotational motor assembly.

In some aspects, the one or more attachment mechanisms comprise openings configured to engage with tabs of the mounting bracket.

In another aspect, the interior surface of the frame comprises a curved inner surface, wherein the rotational motor assembly is generally cylindrical.

In another aspect, the circuit board carrier assembly includes one or more PCB's coupled to the exterior mounting surface.

In some aspects, the frame is generally U-shaped. In other aspects, the frame is generally C-shaped.

In one aspect, the circuit board carrier assembly includes one or more routing channels on the one or more exterior surface configured to receive one or more conduits.

In some aspects, the frame comprises an interior surface portion configured to at least partially conform to a portion of the rotational motor assembly.

In another aspect, the frame is configured to cover at least three sides of the rotational motor assembly.

These and other details and aspects are described herein.

### BRIEF DESCRIPTION OF THE DRAWINGS

FIG. 1 is a side view of an exemplary blood pump that includes an expandable scaffold that supports a blood conduit with an impeller housed therein.

FIG. 2 is a perspective view of a blood pump system that includes the expandable blood pump distal to a handle body within a catheter therebetween, as described herein.

FIG. 3A is a perspective view of handle body operably showing the distal end including a strain relief and sealed housing, as described herein.

FIG. 3B is a perspective view of handle body operably showing the distal end including a strain relief at a proximal end and sealed housing, as described herein.

FIG. 4A and FIG. 4B show an example of a motor assembly locatable within the handle body including a proximal and distal strain relief, as described herein.

FIGS. 5A-5B illustrate an example of an interior arrangement of a motor assembly within a handle body, as described herein.

FIG. 6A and FIG. 6B are detailed views of a motor assembly cradle, as described herein.

### DETAILED DESCRIPTION

The present disclosure is related to medical devices, systems, and methods of use and manufacture. In particular, described herein are pumps adapted to be disposed within a physiologic vessel, wherein the distal pump portion includes one or more components that act upon fluid. For example, the pumps herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

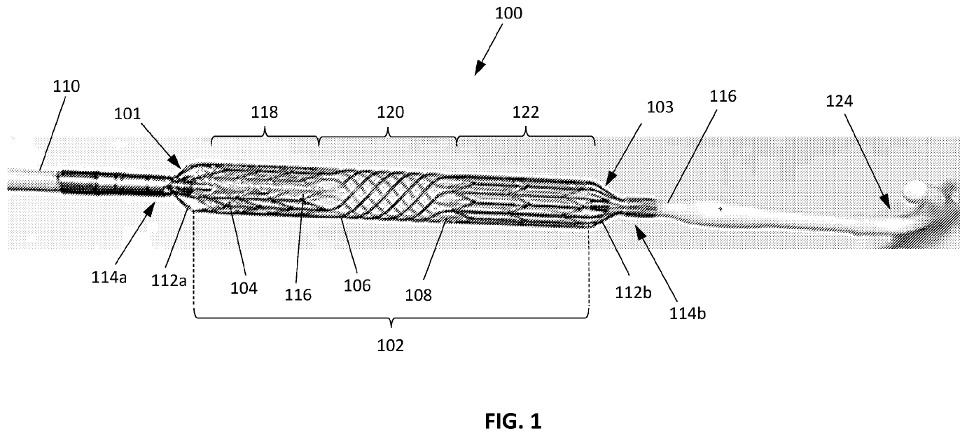

FIG. 1 shows a side view of an exemplary intravascular catheter blood pump 100. The blood pump 100 includes an expandable/collapsible blood conduit 102 that is configured to transition between an expanded state, as shown in FIG. 1, and a collapsed or delivery state (not shown). For example, the conduit 102 may be in the collapsed state when confined within a delivery catheter for delivery to the heart, expanded upon release from the delivery catheter for blood pumping, and collapsed back down within the delivery catheter (or other catheter) for removal from heart. When in the expanded state, the conduit 102 is radially expanded so as to form an inner lumen for passing blood therethrough. When in the expanded state, the inner lumen of the conduit 102 may be configured to accommodate blood pumped by one or more impellers therein. The one or more impellers may be collapsible so that they may collapse to a smaller diameter when the conduit 102 is in the collapsed state. The one or more impellers may be positioned within one or more impeller regions of the conduit 102. In some examples, the impeller region(s) of the conduit 102 is/are radially stiffer than other regions (e.g., adjacent regions) of the conduit 102 to prevent the impeller(s) from contacting the interior walls of the conduit 102.

In this example, the blood pump 100 includes an impeller 104 within a proximal portion of the conduit 102. In some cases, the blood pump 100 can include more than one impeller. For example, the blood pump 100 may include a second impeller in a distal region 122 of the fluid conduit 102. In some cases, blood pump 100 may include more than two impellers. The conduit 102 includes a first (e.g., proximal) end having a first (e.g., proximal) opening 101, and a second (e.g., distal) end having a second (e.g., distal) opening 103. The first opening 101 and second opening 103 may be configured as an inlet and outlet for blood. For example, blood may largely enter the conduit 102 via the second (e.g., distal) opening 103 and exit the conduit 102 via the first (e.g., proximal) opening 101. In such case, the second opening 103 acts as a blood inlet and the first opening 101 acts as a blood outlet. The one or more impellers (e.g., impeller 104) may be configured to pump blood from the inlet toward the outlet. In an exemplary operating position, the second opening 103 (e.g., inlet) may be distal to the aortic valve, in the left ventricle, and the first opening 101 (e.g., outlet) may be proximal to the aortic valve (e.g., in the ascending aorta).

The conduit 102 includes a tubular expandable/collapsible scaffold 106 that provides structural support for a membrane 108 that covers at least a portion of inner surfaces and/or outer surfaces of the scaffold 106. The scaffold 106 includes a material having a pattern of openings with the membrane 108 covering the openings to retain the blood within the lumen of the conduit 102. The scaffold 106 may be unitary and may be made of a single piece of material. For example, the scaffold 106 may be formed by cutting (e.g., laser cutting) a tubular shaped material. Exemplary materials for the scaffold 106 may include one or more of: nitinol, cobalt alloys, and polymers, although other materials may be used.

The blood pump 100 includes proximal struts 112a that extend from the scaffold 106 near the first opening 101 (e.g., blood outlet region) and distal struts 112b that extend from the scaffold 106 near the second opening 103 (e.g., blood inlet region). The proximal struts 112a are coupled to first hub 114a of a proximal shaft 110. The distal struts 112b are coupled to second hub 114b of a distal portion 114. In this example, the first hub 114a includes a bearing assembly through which a central drive cable 116 extends. The drive cable 116 is operationally coupled to and configured to rotate the impeller 104.

In some cases, the impeller 104 is fully positioned axially within the conduit 102. In other cases, a proximal portion of the impeller 104 is positioned at least partially outside of the conduit 102. That is, at least a portion of the impeller may be positioned in axially alignment with a distal portion of the struts 112a.

The conduit 102 and the scaffold 106 may characterized as having a proximal region 118, a central region 120, and a distal region 122. The central region 120 may be configured to be placed across a valve (e.g., aortic valve) such that the proximal region 118 is at least partially within a first heart region (e.g., ascending aorta) and the distal region 122 is at least partially within a second heart region (e.g., left ventricle). The proximal region 118 (and in some cases the distal region 122) may be configured to house an impeller therein. The proximal region 118 may (and in some cases the distal region 122) has a stiffness sufficient to withstand deformation during operation of the blood pump 100 when within the beating heart and to maintain clearance (i.e., a gap) between an impeller region of the blood pump 100 and the rotating impeller 104. The distal region 122 includes the second (e.g., distal) opening 103 of the conduit 102, and may serve as the blood inlet for the conduit 102.

The central region 120 may be less rigid relative to the proximal region 118 (and in some cases the distal region 122). The higher flexibility of the central region 120 may allow the central region 120 to deflect when a lateral force is applied on a side of the conduit 102, for example, as the conduit 102 traverses through the patient's blood vessels and/or within the heart. For example, the central region 120 may be configured to laterally bend upon a lateral force applied to the distal region 122 and/or the proximal region 118. In some cases, it may be desirable for the central region 120 to laterally bend as the conduit 102 traverses the ascending aorta and temporarily assume a bent configuration when the conduit 102 is positioned across an aortic valve. In this example, the central region 120 includes a helical arrangement of longitudinally running elongate elements configured to provide flexibility for lateral bending. In some examples, a distal tip 124 of the blood pump 100 is curved to form an atraumatic tip. In some cases, the distal tip 124 flexible (e.g., laterally bendable) to enhance the atraumatic aspects of the distal tip 124. For example, the distal tip 124 may be sufficiently flexible to bend when pressed against tissue (e.g., by a predetermined amount of force) to prevent puncture of the tissue.

The first hub 114a (e.g., proximal hub) and/or the second hub 114b (e.g., distal hub) may include features that promote smooth blood flow into and/or out of the conduit 102. Such features may prevent or reduce the occurrence of stagnant and/or turbulent blood flow that may otherwise tend to occur in regions near the first opening 101 (e.g., outlet region) and/or the second opening 103 (e.g., inlet region) of the conduit 102. Since stagnant and/or turbulent blood flow is associated with blood coagulation and/or clotting, measures to reduce this can be beneficial to for patient outcome.

In some examples, a distal end or portion of a drive cable can be operably coupled to one or more impellers of the blood pump. A motor assembly can be operationally coupled to or near a proximal end of the drive cable. The motor can be configured to rotate or otherwise control operation of the one or more impellers. For example, the motor assembly can be configured to rotate one or more impellers of the blood pump during use.

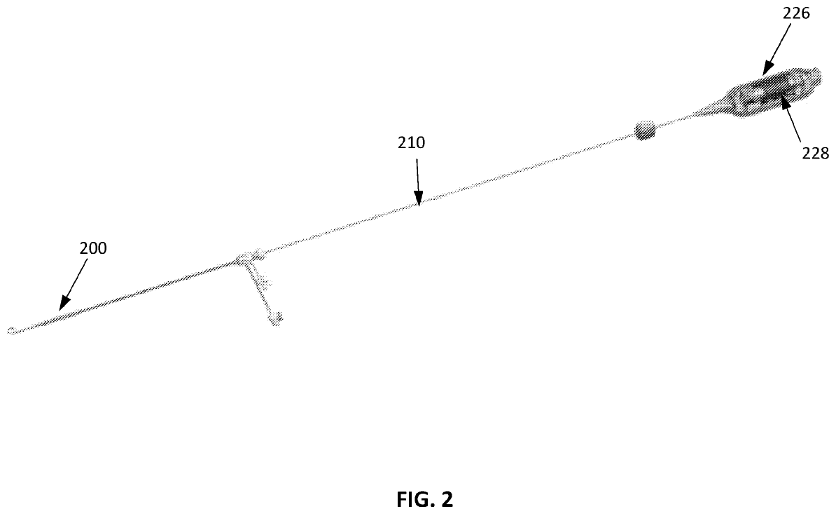

FIG. 2 shows a perspective view of an exemplary intravascular catheter blood pump system having the blood pump 200 positioned distally from a handle 226 having a motor assembly 228 enclosed therein. A drive cable (not shown) may extend through an elongate catheter 210 having one or more layers configured to facilitate rotation and displacement of the blood pump 200 during use. For example, the elongate catheter 210 may have one or more coil layers and/or one or more braid layer configured to translate movement and control of the elongate catheter 210. In some examples, the composition of the elongate catheter 210 can be configured to translate rotation (e.g., torque) from the handle 226 to one or more distal elements (e.g., blood pump) at a 1:1 ratio. In some examples, the composition of the elongate catheter 210 can be configured to translate rotation (e.g., torque) from the motor within the handle 226 to one or more distal elements (e.g., blood pump) at a ratio of handle rotation to distal element rotation greater than 1:1. In some examples, the composition of the elongate catheter 210 can be configured to translate rotation (e.g., torque) from the handle 226 to one or more distal elements (e.g., blood pump) at a ratio of handle rotation to distal element rotation less than 1:1.

In some examples, the drive cable may extend distally from the handle through a lumen of the elongate catheter. The drive-cable catheter may have one or more layers around a central lumen. Each of the one or more layers can be configured to optimize torque response of distal elements relative to rotation of the handle. An elongate catheter may have an outer surface coupled to a distal end of the handle. For example, a drive-cable catheter can have an outer surface fluid sealed to the distal portion of the handle.

In some examples, one or more layers of the elongate catheter may seal against and/or to the handle. For example, an interior of the handle may be fluid sealed, as described herein, and the fluid sealed interior of the handle may continue a fluid seal through an interior lumen of the drive cable housing (e.g., drive-cable catheter).

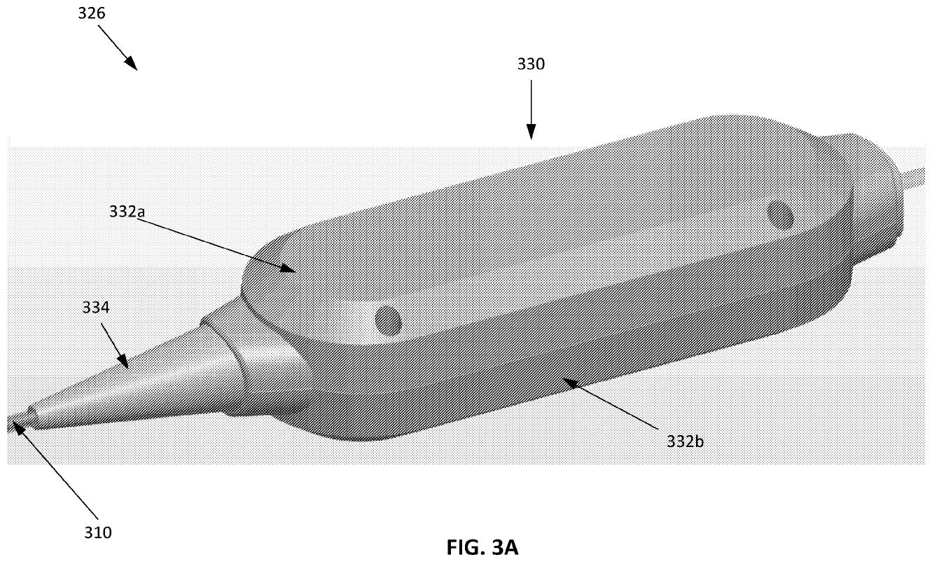

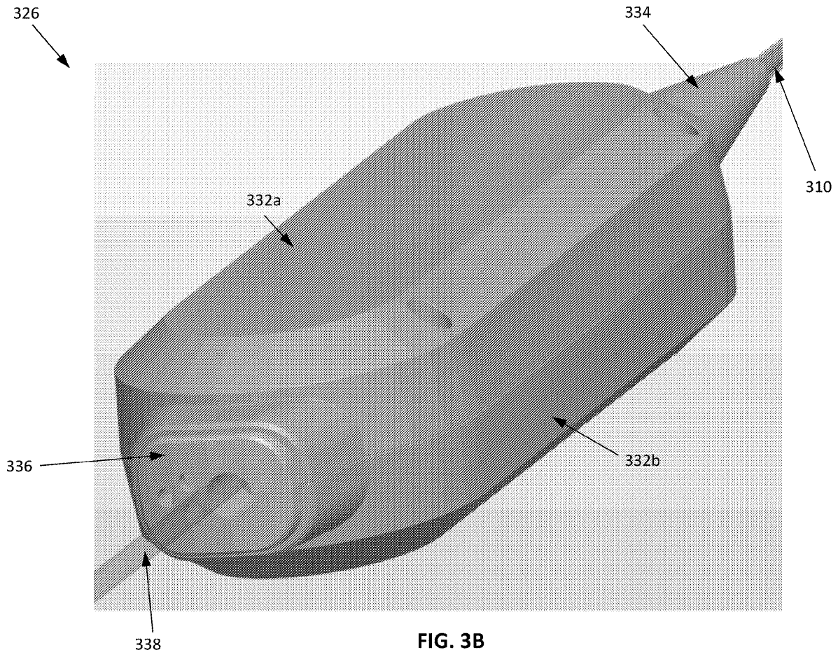

FIG. 3A shows a perspective view of a handle 326 that can be used in operation of an intravascular blood pump. The handle can be positionable outside of a patient during use and may be configured to control or otherwise impact the function, placement, deployment, data acquisition, sensing, etc. of the blood pump and/or associated elements. The handle may have a handle body 330 with one or more portions configured to be coupled to one another. Here, a first handle body portion 332a is coupled (e.g., attached, sealed, affixed, engaged, etc.) to a second handle body portion 332b. In some examples, the housing may have a distal strain relief 334 provided at a distal portion of the handle where the catheter shaft 310 of the blood pump enters/exits the handle. The distal strain relief can facilitate a connection and/or transition of the catheter shaft 310 to motor and fluid connections within the handle 326.

FIG. 3B shows a proximal perspective view of a handle 326 that can be used in operation of an intravascular blood pump. In this example, the handle body has a first handle body portion 332a coupled to a second handle body portion 332b. A distal strain relief 334 is shown at an opposite (distal) end of the handle body from a proximal strain relief 336. In some examples, the handle body may have a plurality of strain reliefs that can be configured to facilitate one or more connections and/or transitions of elements to and/or from an interior of the handle body to an exterior of the handle. In both FIGS. 3A and 3B, an elongate member is shown at least entering each strain relief at a distal portion of the handle. For example, the catheter shaft 310 may engage or enter the handle body through the distal strain relief 334. In some examples, proximal connections 338 such as electrical connections (e.g., power cables, control cables, and/or data transfer cables) or fluid connections (e.g., saline flush) may engage, couple to, transition, or otherwise contact or enter the handle via the proximal strain relief 336. While only a single proximal connection 338 is shown entering the handle via proximal strain relief 336, it should be understood that multiple proximal connections may be implemented in or accommodated by the handle and proximal strain relief.

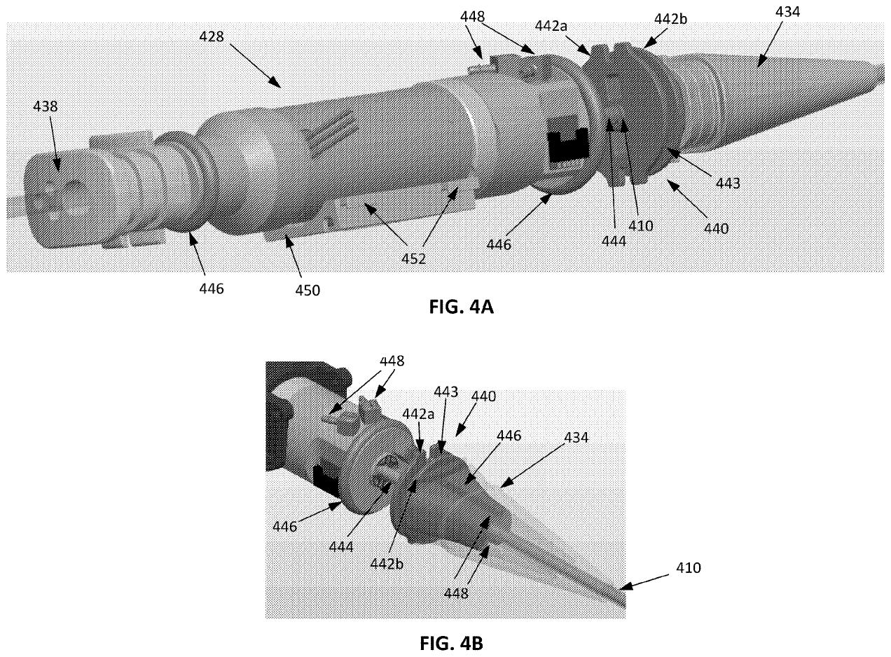

FIGS. 4A-4B show cutaway views of a motor assembly 428 with the handle body not shown for ease of illustration. The distal strain relief 434 can include or be coupled to a strain relief coupling member 440. The strain relief coupling members described herein may be configured to increase structural rigidity of the strain relief assembly and provide a coupling or attachment point between the strain relief and the handle body. In some examples, strain relief coupling member may include an inner coupling portion 442a that can be more rigid (e.g., less flexible) than an outer coupling portion 442b. In other examples, the inner and outer coupling portions 442a/442b can comprise the same material rigidity, but can be more stiff or less flexible/compliant than the distal strain relief 434. The inner and outer coupling portions 442a/442 can be generally disc shaped, as shown, and can include a gap 443 between the inner coupling portion 442a and the outer coupling portion 442b. In some embodiments, a handle body engagement feature is configured to be placed within the gap 443 between the inner and outer coupling portions to provide a rigid attachment or coupling between the coupling member 440 and the handle body (described in more detail below). Another disc shaped member or surface may be disposed within the gap to allow the coupling member to be rotatable relative to the handle body when the coupling member is coupled or attached to the handle body. For example, the disc shaped member in the gap 443 may have a radius that is smaller than a radius of both the inner and outer coupling portions 442a/442b. In some examples, the strain relief 434 and/or strain relief coupling member 440 may be rotatable (e.g., adjustable) relative to the handle body. As described above, the gap 443 of the coupling member 440 may include a recessed disc or rounded surface that is rotatable at the handle body engagement.

As shown in FIGS. 4A-4B, both the strain relief 434 and the coupling member 440 can comprise lumens or openings configured to receive a shaft or elongate member. In FIG. 4B, catheter shaft 410 of the catheter blood pump can be seen entering a distal end of the strain relief 434 and passing through the strain relief 434 before entering the coupling member 440. A lumen of the strain relief 434 can be in communication with a lumen of the coupling member. In FIG. 4A, the catheter shaft 410 can be seen extending out of the proximal portion of the coupling member 440. In this illustration, blood pump drive cable 444 is shown exiting from the catheter shaft 410 towards the motor assembly 428. As described above, the motor assembly is configured to impart rotation upon the drive cable 444 to rotate one or more impellers located in the catheter blood pump. While the illustrated embodiment shows the catheter shaft extending through both the strain relief and the coupling member, but terminating prior to the motor assembly, it should be understood that other embodiments can have the catheter shaft extending further proximally (e.g., towards the motor assembly) or alternatively not extending fully through the coupling member. However, it should be understood that in the illustrated embodiment, the rigidity provided by the coupling member can provide additional support and strain relief towards the catheter shaft, including specifically at the junction where the catheter shaft ends, and the drive cable extends proximally towards the motor assembly.

The strain relief 434 of FIGS. 4A-4B may comprise a flexible or compliant material (e.g., a semi-rigid but conformable material) such as a silicone, polymer, urethane, etc. configured to reduce, prevent, inhibit, or shield against bending, kinking, or folding forces that may act on an elongate member passing therethrough, such as the catheter shaft and/or drive cable of the catheter blood pump. The strain relief coupling member 440 may also comprise a flexible or compliant member such as silicone, polymer, urethane, etc. Alternatively, the strain relief coupling member may comprise a rigid material. In some embodiments, the strain relief 434 is more flexible than the coupling member. For example, the coupling member 440 may comprise a rigid or semi rigid polymer, plastic, or thermoplastic designed and configured to provide a structural connection point to the handle body and a rigid support for elongate members such as the catheter shaft and/or drive cable, while the strain relief 434 may comprise a flexible urethane, polymer, or silicon configured to allow some bending or deflection of the elongate member(s) while preventing kinking or sharp bending of the elongate member(s). The strain relief coupling member 440 can be operably coupled or attached to the strain relief. In some embodiments, the strain relief 434 is placed over a distal portion 446 of the coupling member 440. The strain relief can be overmolded on the distal portion of the coupling member, for example. In some embodiments, the coupling member can include one or more engagement channels or features 448 configured to interact or engage with the strain relief, such as during overmolding, to ensure a solid connection or increase engagement between the strain relief and the coupling member and prevent the strain relief from detaching from the coupling member during use. In some embodiments, the coupling member 440 comprises a first molded polymer, and the strain relief 434 comprises a second molded polymer. In some examples, the coupling member and the strain relief comprise co-molded polymers. As described above, the coupling member can have more stiffness (or less flexibility or compliance) than the strain relief.

In FIG. 4B, a distal end of the strain relief coupling member 440 is shown coupled to a proximal end of the strain relief 315 such that the drive cable 444 and/or the catheter shaft 410 can be configured to extend from inside of the handle body to an exterior of the handle body through the strain relief and the coupling member.

One or more toroidal elements 446 may be coupled to the motor assembly 428 and can be configured to provide support and/or a contact point for mounting of the motor assembly 428 to an interior of the handle. In some examples, a toroidal element may be an o-ring configured to dampen, reduce, prevent, or eliminate vibration of the motor assembly during operation. In some examples, one or more o-rings may be positioned around the motor assembly to create or support a fluidic seal between the handle body and/or the motor assembly within the handle body.

The motor assembly 428 may include fluid flow through one or more elements of the motor assembly. FIGS. 4A-4B include fluid couplers 448 that can be configured to engage tubing and direct a flow of fluid through the tubing and the motor assembly. In some examples, there can be fluid inlet couplers and/or fluid outlet couplers configured to direct a flow of fluid into and out of the motor assembly, respectively. The fluid couplers can also provide a flow of fluid to the catheter pump via the catheter shaft, such as for purging fluid during a cardiac support procedure.

In some examples, fluid flowing through the motor assembly (e.g., through the fluid couplers) can be configured to prime the motor assembly (e.g., a motor). For example, a fluid may flow through tubing routed within the handle body and to displace air within a motor and prime the motor for operation. In some examples, the motor assembly may comprise a pump (e.g., a centrifugal pump) that can be configured to receive (e.g., pump) a fluid configured to prime the pump for operation. In some examples, the motor assembly may comprise a fluid displacement pump configured to receive (e.g., pump) a fluid configured to prime the pump for operation.

In some examples, fluid flowing through the motor assembly (e.g., through the fluid couplers) can be configured to lubricate the motor assembly (e.g., a motor). For example, a lubricious fluid may be routed through the handle body to one or more fluid couplers. A lubricating fluid may be configured to adjust the lubricity of one or more surfaces of the motor assembly. For example, a lubricating fluid may be introduced to the motor assembly and contact (e.g., coat) one or more surfaces of the motor to reduce friction between the contacted surface and one or more elements of the motor assembly.

In some examples, fluid flowing through the motor assembly may be configured to flush one or more elements of a blood pump system. For example, a fluid configured to flush the blood pump may flow through the handle body (e.g., through tubing routed through the handle body) and continue distally from the motor assembly through an elongate member (e.g., catheter) to a blood pump positioned at a distal end. The fluid may be configured to flush the blood pump during use. In some examples, a fluid configured to flush a blood pump system can be saline, water (e.g., sterile water), or other fluid configured to flush (e.g., flow) through an elongate member from the handle body to a blood pump located within a patient.

In some examples, more than one fluid may flow through the handle body (e.g., through tubing routed within the handle body) and the motor assembly. For example, a priming fluid, flush fluid, lubricating fluid, body fluid (e.g., blood) may be configured to flow through, within, etc. the handle body. In some examples, tubing routed through (e.g., within) the handle body (e.g., motor assembly) can be configured to receive one or more different types of fluids. In some examples, tubing routed through (e.g., within) the handle body (e.g., motor assembly) can be configured to receive the same type of fluid. In some examples, tubing routed through (e.g., within) the handle body (e.g., motor assembly) can be configured to receive a combination of one or more types of fluid. In some examples, a first type of fluid may be routed via tubing through the handle body and one or more additional fluids may be routed via different tubing through (e.g., within) the handle body (e.g., motor assembly). For example, a first fluid routing system may comprise a tube routing a first type of fluid therethrough and a second fluid routing system may comprise a separate tube routing the second fluid therethrough, etc.

Referring still to FIG. 4A, the motor assembly 428 can include a mounting platform 450 with one or more engagement features 452 configured to be attached to a cradle assembly (described below). The mounting platform can be integrated into the motor assembly 428, or alternatively can be fixedly coupled or attached to the motor assembly (e.g., with adhesive or screws). The engagement features 452 may include, for example, clips or tabs configured to engage with a corresponding engagement feature (e.g., an opening) on the cradle assembly. The cradle assembly can provide a mounting location for electronics, sensors, or other sensitive components within the handle.

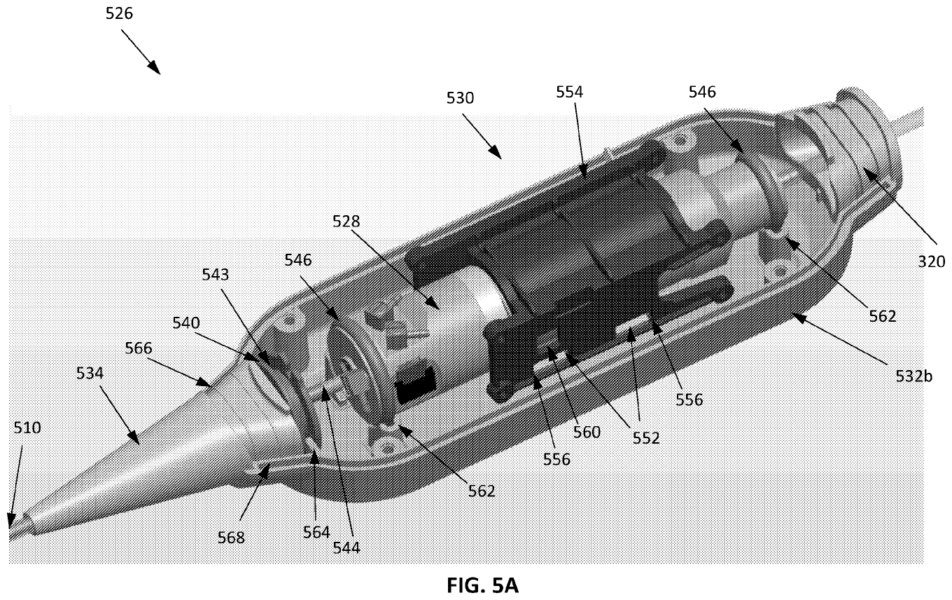

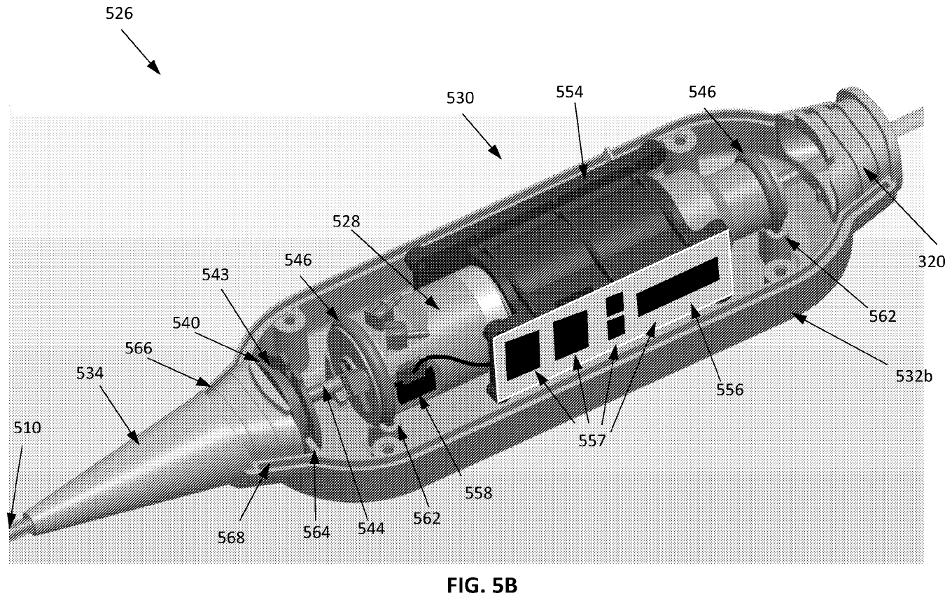

FIG. 5A shows a view of handle 526 that includes a handle body 530 and a motor assembly 528 mounted within the handle body. This view also shows cradle assembly 554 mounted on the motor assembly 528. Tabs 552 of the motor assembly mounting bracket can be seen extending through and engaging with openings 556 in the cradle assembly. The cradle assembly 554 may be configured to optimize attachment, organization, routing, orientation, positioning, operation, etc. of components within the handle. In some examples, the cradle assembly 554 can be configured to receive additional elements within the handle body. For example, the cradle assembly can provide for one or more mounting surfaces configured to receive a printed circuit board (PCB) thereon.

FIG. 5A shows the cradle assembly 554 mounted onto the motor assembly 528 (via the mounting platform) without a PCB mounted thereon, and FIG. 5B shows the cradle assembly 554 with at least one PCB 556 mounted thereon. The PCB can include, for example, one or more processors, electrical components, or chip components 557 disposed thereon. In FIG. 5B, an electrical connection 558 on the motor assembly can be electrically connected to the PCB 556. In some examples, the electrical connection between the PCB and (e.g., a wire) can pass through one or more openings 560 (FIG. 5A) on the cradle assembly. The PCB and electrical components disposed thereon can control operation of the motor assembly and other electrical components of the catheter blood pump. The PCB and electrical components can receive input or control instructions from the user, and control the blood pump accordingly. In some embodiments, the PCB and electrical components are electrically coupled to sensors on or within the catheter blood pump and/or the motor assembly, including but not limited to pressure sensors, flow sensors, or rotational encoders/sensors (e.g., for measuring rotation of the motor assembly or drive cable).

The handle body 530 can further include mounting posts or elements 562 configured to support the motor assembly 528. In some embodiments, the mounting posts or elements 562 can be integrated into the handle body, such as integrated within the handle body portion 532b. In some examples, the mounting posts or elements 562 can directly contact the motor assembly. In other embodiments, they can contact toroidal elements or o-rings 546 to reduce vibrations imparted by the motor assembly 528 on the handle 526. In the illustrated embodiment, the mounting posts or elements 562 are positioned near a proximal and distal end of the motor assembly 528, to provide support for the motor assembly within the handle.

FIGS. 5A-5B also show the attachment or coupling between the strain relief 534/strain relief coupling member 540 and the handle body 530. As shown, the handle body can include extensions or tabs 564 configured to reside within the gap 543 of the strain relief coupling member 540. The coupling member may be retained within the handle body with extensions or tabs 564 while still allowing for rotation of the strain relief relative to the handle body (e.g., allowing for rotation of the extensions or tabs within the gap).

As shown, the strain relief 534 can also provide a fluidic seal between an interior and exterior of the handle body. For example, the strain relief may include one or more o-rings, raised portions, or sealing members 566 configured to engage with an interior of the handle body and assist in retaining the strain relief to the handle body. Contact between the handle body and the strain relief/strain relief sealing members can be configured to maintain a fluid seal for the interior of the handle body.

As described herein, the handle body can be fluidically sealed. One or more gaskets may be locatable between contacting surfaces of the handle body portions. For example, in FIG. 5A, channel 568 is shown generally around a perimeter of the second handle body portion 532b and can be configured to receive a first handle body portion in a fluid-sealed engagement. In some examples, the channel 568 can be configured to receive a gasket configured to seal contact between handle body portions. Additional structures of the handle body, motor assembly, strain reliefs, etc. can be configured to facilitate and promote a fluid seal of the handle body. For example, strain reliefs can be configured to receive one or more handle body portions in a fluid seal engagement.

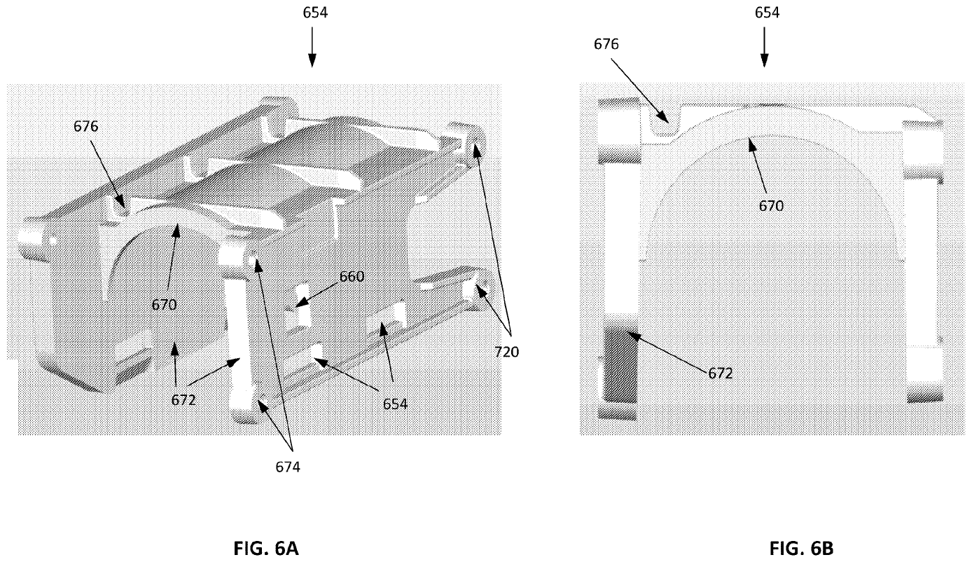

FIGS. 6A-6B show additional details of a cradle assembly 654 that can comprise a frame. In these examples, a portion of an interior surface 670 of the cradle assembly 654 is configured to conform to and engage at least a portion of the motor assembly. For example, the interior surface 670 can include a curvature configured to correspond to the shape (e.g., a curvature) of an exterior portion of the motor assembly. The interior curve of the cradle assembly interior surface portion can extend between one or more sidewalls 672. In some examples, a motor assembly cradle can be configured to engage a motor assembly around a portion of the motor assembly exterior perimeter to allow for easy removal of the cradle from the motor assembly. In some examples, the cradle assembly can be shaped to facilitate easy installation and removal of the motor assembly from the cradle. For example, the cradle assembly can comprise a generally U-shaped or C-shaped design in which the frame can slide over a motor assembly for attachment to the motor assembly mounting bracket. The cradle assembly can be configured to cover at least three sides of the motor assembly when the cradle assembly is mounted to the motor assembly.

FIG. 6A shows one or more mounting points 674 where a circuit board (e.g., PCB) may be attached, such as with screws or rivets. Also shown are openings or attachment points 654 for attachment to the motor assembly mounting platform, and opening 660 for electrical connection between the motor assembly and the PCB. Additionally, the cradle assembly can be configured to route one or more conduits, tubing, wires, etc. within the handle body. One or more channels 676 may be positioned on the cradle and configured to receive a length of conduit, tubing, wire, etc. therethrough.

The handle assemblies described herein may include one or more sensors, which may be in electrical communication or coupling to the PCB, motor assembly, or other system components as described herein. One or more sensors may be configured to acquire data associated with an intravascular blood pump. In some examples, one or more sensors may be in operable communication with the motor assembly. For example, a flow of fluid may be directed through the handle (e.g., through the motor assembly) and one or more sensors may be configured to sense (e.g., acquire) attributes of the fluid flowing therethrough.

One or more sensors may be a pressure sensor configured to sense a pressure of fluid flowing through the intravascular blood pump assembly. One or more pressure sensors may be locatable on, in or near the motor assembly and configured to acquire attributes of the fluid flowing through the motor assembly. For example, one or more pressure sensors may be configured to sense a pressure of fluid flowing through the motor assembly.

In some examples, one or more pressure sensors can be configured to detect purge pressure. For example, a catheter purge line may engage a fluid coupler and route fluid through the motor assembly while the one or more pressure sensors can be configured to detect a leak, air, flow, etc. through the catheter purge line.

One or more sensors may be a flow rate sensors configured to sense a flow rate of a fluid flowing through an intravascular blood pump assembly. For example, one or more flow-rate sensors may be locatable on, in, or near the motor assembly and configured to acquire a flow rate of a fluid passing through the motor assembly. An example of one or more flow rate sensors may be one or more sensors in operable communication with a flow of blood through the motor assembly.

In some examples, one or more sensors (e.g., flow rate sensor, pressure sensor, etc.) can be locatable on and/or in one or more of the fluid couplers, as described herein. For example, one or more sensors may be operable connected to one or more of the fluid couplers of the motor assembly and configured to sense (e.g., detect) attributes (e.g., pressure, flow rate, temperature, etc.) of a fluid therein and/or flowing therethrough.

In some examples, one or more sensors (e.g., pressure sensor) can be locatable on a fluid coupler (e.g., an inlet fluid coupler and/or an outlet fluid coupler) and may be configured to measure a fluid column pressure. In some examples, the measured fluid column pressure may be configured and/or used to estimate a pressure blood pump (e.g., at a proximal hub of the blood pump system).

One or more sensors may be motor assembly operation sensors configured to detect (e.g., sense) attributes of the motor assembly during operation. For example, one or more sensors may be configured to detect an operating temperature of the motor assembly. One or more sensors may be configured to detect the number of rotations made by the motor assembly. In some examples, one or more sensors may be configured to detect the number of rotations of the drive cable.

The handle body may enclose electrical circuitry. Electrical circuity (e.g., circuit boards) can be configured to operationally coordinate electrical connection between one or more elements of the intravascular blood pump system. In some examples, printed circuit boards (PCB) may be configured to coordinate electrical communication with the motor assembly, one or more sensors, one or more power sources, one or more communication systems, a controller, etc. In some examples, peripheral components may be in electrical communication with the motor assembly as part of an electrical circuit. In some examples, data detected, acquired, and/or generated by the intravascular blood pump system (e.g., motor assembly) may be transmitted to one or more external devices. For example, a communication means may be operably coupled to the motor assembly (e.g., to the one or more sensors) and be configured to send and receive data between the intravascular blood pump system and a remote device. In some examples, the communication means is a wireless communication means configured to wireless transmit and/or receive data, operation input, or otherwise control or evaluate the operation of the intravascular blood pump system (e.g., motor assembly).

In some examples, a handle as described herein, may receive one or more connections to one or more peripheral components. Some examples of peripheral components may be computing devices, sensors, controllers, containers, power sources, conduits, etc. In some examples, a peripheral component may be any component outside of the handle used with an intravascular blood pump or associated procedure.

Methods of advancing and operating a blood pump system, as described herein can include advancing a blood pump into a blood vessel of a patient. In some examples, the blood pump can be a self-expanding blood pump operably coupled to a distal end of a catheter. The self-expanding blood pump may be inserted into a sheath that can be passed through an introducer into a blood vessel of a patient. The sheath may be configured to compress or retain the blood pump in a compressed state during navigation and advancement through the patient's blood vessel. A catheter (e.g., drive cable catheter) may extend proximally from the proximal end of a blood pump to a handle body. As described herein, the drive cable shaft may be operably coupled to a motor assembly within the handle body and extend distally to an impeller within the blood pump. In some examples, the blood pump is positioned at a location within the blood vessel for operation. The blood pump may be advanced from the sheath and expand to a deployed state.

In some examples, the drive cable catheter can be configured to facilitate advancement and maneuverability of the blood pump through the blood vessel. For example, the drive cable catheter can be configured to translate rotation from a user (e.g., healthcare professional) via engagement and rotation of the handle. For example, navigating and advancing the blood pump through the patient's vasculature may require adjustment of orientation of the distal end of the blood pump. Accordingly, the handle may be rotated such that the distal end (e.g., the blood pump) rotates in a corresponding manner for accommodate the necessary adjustment and proceed with the placement, position, or advancement of blood pump within the patient's vasculature.

The handle may be used to control operation of the blood pump during use. For example, a controller within the handle or associated with a remote device may be configured to control one or more functions of the blood pump (e.g., rate of rotation, initiation, termination, detecting with one or more sensors, etc.).

An example of a process by which a blood pump system, as described herein, may be introduced, advanced, and operated within the patient's vasculature. The blood pump (e.g., self-expanding blood pump) may be sheathed and introduced into the patient's vasculature. In some examples, an introducer may be employed to facilitate a transition of the blood pump into the patient's blood vessel. The blood pump (e.g., sheathing catheter with the compressed blood pump therein) may then be advanced into the patient's vasculature. In some examples, a user may advance or navigate advancement of the blood pump using the handle body. For example, advancing the blood pump distally into the blood vessel may require adjustment to accommodate an obstruction or junction in the blood vessel. Accordingly, the handle may be rotated, and the drive cable catheter may translate the rotation (e.g., torque) from the handle to the distal end (e.g., the blood pump) until the advancement of the sheathing catheter can continue. In some examples, positioning of the blood pump may be optimized or otherwise require rotation for proper placement. Such rotation may also be facilitated by rotating the handle.

Any of the blood pumps described herein may include surfaces with one or more anticoagulant agents. For example, at least a portion of one or more of the hubs, conduits (e.g., scaffold and/or membrane), struts (e.g., proximal and/or distal struts), distal tips and/or impellers of the blood pumps described herein may include a coating or material having an anticoagulant agent. In some cases, the anticoagulant agents may include drugs such as heparin, warfarin and/or prostaglandins.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. A catheter handle device, comprising:
a handle body; and
a strain relief assembly partially disposed within the handle body, the strain relief assembly providing access for an elongate member to pass from an interior of the handle body to an exterior of the handle body, the strain relief assembly comprising a coupling member that is attached to the handle body and a strain relief member attached to the coupling member and extending from the handle body, wherein the strain relief member is more flexible than the coupling member.

2. The device of claim 1, wherein the strain relief member comprises a first polymer and the coupling member comprises a second polymer.

3. The device of claim 1, wherein the strain relief member is molded over a distal portion of the coupling member.

4. The device of claim 1, wherein the coupling member comprises a first coupling portion and a second coupling portion separated by a gap.

5. The device of claim 4, wherein the gap of the coupling member is configured to receive a tab or extension of the handle body.

6. The device of claim 5, wherein the strain relief assembly is rotatable within the handle body when the tab or extension of the handle body is inserted into the gap of the coupling member.

7. The device of claim 6, wherein the gap includes an inner surface that is disc-shaped to facilitate rotation of the strain relief assembly.

8. The device of claim 1, wherein the strain relief member forms a fluidic seal with an interior of the handle body.

9. The device of claim 1, wherein the strain relief member includes one or more sealing elements.

10. The device of claim 9, wherein the sealing elements comprise raised portions or o-rings.

11. The device of claim 1, wherein a lumen of the coupling member is in communication with a lumen of the strain relief member.

12. The device of claim 1, wherein the strain relief member is a flexible or compliant material and the coupling member is a substantially rigid material.

13. The device of claim 1, wherein the elongate member comprises a catheter shaft.

14. The device of claim 1, wherein the elongate member comprises a drive cable of a motor assembly.

15. The device of claim 1, wherein the handle body comprises a clamshell design with a first handle body portion coupled to a second handle body portion.

16. The device of claim 15, wherein an o-ring is provided between the first and second handle body portions to fluidically seal the handle body portion.

17. The device of claim 1, wherein the handle body further comprises one or more mounting elements configured to support a motor assembly.

18. The device of claim 17, wherein the one or more mounting elements comprises a first mounting element configured to support a distal portion of the motor assembly and a second mounting element configured to support a proximal portion of the motor assembly.

19. The device of claim 17, further comprising a motor assembly disposed in the handle body and supported by the one or more mounting elements.

20. The device of claim 19, further comprising one or more o-rings disposed around the motor assembly and configured to contact the one or more mounting elements to reduce vibration imparted by the motor assembly on the handle body.

21. The device of claim 19, further comprising a cradle assembly removably coupled to the motor assembly, the cradle assembly comprising at least one printed circuit board (PCB) disposed on the cradle assembly.

22. The device of claim 21, wherein the cradle assembly is configured to at least partially conform to an outer surface of the motor assembly.

23. The device of claim 21, wherein the PCB further comprises electronics configured to control operation of the motor assembly.

24. The device of claim 23, further comprising an electrical connection on the motor assembly configured to be electrically coupled to the PCB.

25. The device of claim 24, wherein the electrical connection passes through an opening on the cradle assembly.

26. The device of claim 1, wherein the strain relief assembly and the coupling assembly are co-molded polymers.

27.-54. (canceled)
